(19) United States
(12) Patent Application Publication
(10) Pub. No.: US 2024/0032871 A1
(43) Pub. Date: Feb. 1, 2024

(54) Digital filtering method for photoplethysmography device

(71) Applicant: REALTEK SEMICONDUCTOR CORPORATION, Hsinchu (TW)

(72) Inventor: MENG-HSUAN WU, Hsinchu (TW)

(21) Appl. No.: 18/215,190

(22) Filed: Jun. 28, 2023

(30) Foreign Application Priority Data
Jul. 26, 2022 (TW) 111128022

(51) Int. Cl. A61B 5/00; A61B 5/024

(52) U.S. Cl. CPC A61B 5/7203; A61B 5/02416

(57) ABSTRACT

A digital filtering method is applicable to a photoplethysmography (PPG) device. The PPG device samples a mixed-light signal M time(s) to obtain M mixed-light digital value(s), and samples an ambient-light signal N time(s) to obtain N ambient-light digital value(s), wherein each mixed-light digital value includes a controllable-light component and an ambient-light component. The method includes: preparing a digital filter whose filter order is (M+N−1); using the digital filter to multiply the M mixed-light digital value(s) by M coefficient(s) respectively and thereby generate M value(s); using the digital filter to multiply N ambient-light digital value(s) by N coefficient(s) respectively and thereby generate N value(s); and using the digital filter to add up the M value(s) and the N value(s) and thereby generate an output value.

### BACKGROUND OF THE INVENTION

### 1. Field of the Invention

The present disclosure relates to a digital filtering method, especially to a digital filtering method for a photoplethysmography (PPG) device.

### 2. Description of Related Art

The photoplethysmography (PPG) technology involves illuminating skin with a controllable light source (e.g., a light emitting diode (LED)) and measuring the consequent variation in optical absorption, and thus can be applied to multiple kinds of applications (e.g., the measurement of heartbeat and blood oxygen). However, in addition to the controllable light source, other light sources (e.g., sunlight and indoor light) usually exist in the same space, and the influence of these light sources (a.k.a. ambient light) should be eliminated to ensure the accuracy of the measurement of the variation in optical absorption. The ambient light can be filtered out with analog circuits or hardware, but it is difficult to implement a multi-order filter with analog circuits or hardware.

### SUMMARY OF THE INVENTION

An object of the present disclosure is to provide a digital filtering method for a photoplethysmography (PPG) device. This digital filtering method can easily eliminate ambient light in a multi-order filtering manner.

An embodiment of the digital filtering method of the present disclosure is applicable to a PPG device. The PPG device is configured to sample a mixed-light signal M time(s) in turn to obtain M mixed-light digital value(s) and is configured to sample an ambient-light signal N time(s) in turn to obtain N ambient-light digital value(s), wherein both the M and the N are positive integers, and each of the M mixed-light digital value(s) includes a controllable-light component and an ambient-light component. The embodiment includes the following steps: preparing a digital filter, wherein a number of the order of the digital filter is (M+N−1); using the digital filter to multiply the M mixed-light digital value(s) by M coefficient(s) respectively in accordance with the sequence of the M mixed-light digital value(s) being obtained, and thereby generating M value(s); using the digital filter to multiply the N ambient-light digital value(s) by N coefficient(s) respectively in accordance with the sequence of the N ambient-light digital value(s) being obtained, and thereby generating N value(s); and using the digital filter to add up the M value(s) and the N value(s), and thereby generating an output value.

These and other objectives of the present invention will no doubt become obvious to those of ordinary skill in the art after reading the following detailed description of the preferred embodiments that are illustrated in the various figures and drawings.

### BRIEF DESCRIPTION OF THE DRAWINGS

FIG. 1 shows a photoplethysmography (PPG) device including an analog front-end capable of performing the digital filtering method of the present disclosure.

FIG. 2 shows an embodiment of the digital filtering method of the present disclosure.

FIG. 3 shows the PPG device of FIG. 1 performing a Z-domain sampling operation through the time domain.

FIG. 4a shows that two ambient-light digital values are between two closest mixed-light digital values on the timeline due to the control of a controllable light source.

FIG. 4b shows that two mixed-light digital values are between two closest ambient-light digital values on the timeline due to the control of a controllable light source.

FIG. 5a shows that one ambient-light digital value is between two closest mixed-light digital values on the timeline due to the control of a controllable light source.

FIG. 5b shows that no ambient-light digital value is between two closest mixed-light digital values on the timeline due to the control of a controllable light source.

FIG. 6a shows that one mixed-light digital value is between two closest ambient-light digital values on the timeline due to the control of a controllable light source.

FIG. 6b shows that no mixed-light digital value is between two closest ambient-light digital values on the timeline due to the control of a controllable light source.

### DETAILED DESCRIPTION OF THE PREFERRED EMBODIMENTS

The preset specification discloses a digital filtering method for a photoplethysmography (PPG) device. The digital filtering method can easily eliminate ambient light in a multi-order filtering manner.

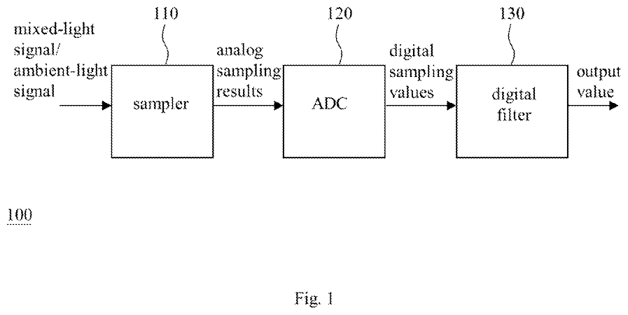

FIG. 1 shows a PPG device 100 including an analog front-end capable of performing the digital filtering method of the present disclosure. The PPG device 100 includes a sampler 110 (e.g., a device including a photo diode (PD) and a transimpedance amplifier (TIA)), an analog-to-digital converter (ADC) 120, and a digital filter 130 (e.g., a finite impulse response (FIR)). Since the PPG device 100 itself does not concern the main features of the present invention and can be implemented with known/self-developed technologies, the detail of the PPG device 100 is omitted here. It is noted that the coefficient(s) of the digital filter 130 can be determined in a known/self-developed manner; for example, if the digital filter 130 is an FIR filter, the coefficient(s) of the FIR filter can be calculated by performing the inverse Fourier transform (IFT) to a designate frequency response, wherein the designate frequency response relates to the specification of the PPG device 100.

Referring to FIG. 1, the PPG device 100 samples a mixed-light signal at least M time(s) to obtain M mixed-light digital value(s) in turn and samples an ambient-light signal at least N time(s) to obtain N ambient-light digital value(s) in turn; afterward, the PPG device 100 filters all of the above-mentioned digital values in a digital manner and thereby generates an output value, wherein both the M and the N are positive integers. In detail, the sampler 110 samples the mixed-light signal M time(s) or more than M times to generate M mixed-light analog sampling result(s) in turn, and the sampler 110 also samples the ambient-light signal N time(s) or more than N times to generate N ambient-light analog sampling result(s) in turn; for example, a PD of the sampler 110 samples the mixed-light/ambient-light signal to generate a photoelectric current, and then a TIA of the sampler 110 converts the photoelectric current into a voltage signal and outputs (N+M) voltages of the voltage signal as the (N+M) analog sampling results. In addition, the ADC 120 performs analog-to-digital conversion M time(s) to convert the M mixed-light analog sampling result(s) into the M mixed-light digital value(s), and the ADC 120 also performs analog-to-digital conversion N time(s) to convert the N ambient-light analog sampling result(s) into the N ambient-light digital value(s). Furthermore, the digital filter 130 processes the M mixed-light digital value(s) and the N ambient light digital value(s) to generate the output value.

It is noted that the mixed-light signal includes a controllable light signal (e.g., the light signal of an LED) and the ambient light signal, and consequently each of the M mixed-light digital value(s) includes a controllable-light component and an ambient-light component. The controllable-light component is usually originated from at least one controllable light source (e.g., at least one LED) of a transmitter of the PPG device 100 while the ambient-light component varies with the variation in the environment, wherein the variation in the environment may be caused by the posture, motion, etc., of a user using the PPG device 100. When the at least one controllable light source is turned on, the sampler 110 samples the mixed-light signal; and when the at least one controllable light source is turned off, the sampler 110 samples the ambient light signal. It is also noted that the sampling frequency of the PPG device 100 is usually not lower than 100 kHz; for example, the sampling frequency of the PPG device 100 is 500 kHz, which means that the sampling cycle of the PPG device 100 is 2 μs, but this is not a limitation in the implementations of the present invention.

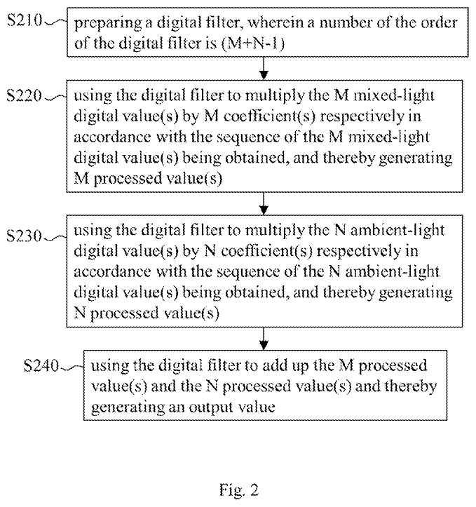

FIG. 2 shows an embodiment of the digital filtering method of the present disclosure. This embodiment includes the following steps:
S210: preparing a digital filter (e.g., an FIR filter), wherein a number of the order of the digital filter is (M+N−1). The M and the N are defined in the preceding paragraph.
S220: using the digital filter to multiply the M mixed-light digital value(s) by M coefficient(s) respectively in accordance with the sequence of the M mixed-light digital value(s) being obtained, and thereby generating M value(s). In an exemplary implementation, provided that the variation in the ambient light is faster than the variation in the physiological signal, the digital filter is a high pass filter (HPF) for filtering out the variation in the ambient light. In an exemplary implementation, the M coefficient(s) is/are predetermined and/or adjustable. In an exemplary implementation, the sign of the M coefficient(s) is a first sign, which means that the sign of the M value(s) is the first sign, wherein the first sign is a positive sign or a negative sign according to the demand for implementations.
S230: using the digital filter to multiply the N ambient-light digital value(s) by N coefficient(s) respectively in accordance with the sequence of the N ambient-light digital value(s) being obtained, and thereby generating N value(s). In an exemplary implementation, the N coefficient(s) is/are predetermined and/or adjustable. In an exemplary implementation, the sign of the N coefficient(s) is a second sign, which means that the sign of the N value(s) is the second sign, wherein the second sign is opposite to the first sign; for example, the second sign is a negative/positive sign when the first sign is a positive/negative sign.
S240: using the digital filter to add up the M value(s) and the N value(s) and thereby generating an output value. It is noted that the ambient-light component ratio

$$( i . e . , \frac{ambient - light component}{controllable - light component} )$$

of the output value is usually negligible, which means that the ambient light cancellation (ALC) effect of the present embodiment is usually excellent. For example, the measurement of the ALC effect of a conventional prior art is −70 dB while the measurement of the ALC effect of the present embodiment is between −76 dB and −82 dB. The ALC effect of the present invention is not limited to the above example.
It is noted that the execution of the steps S220-S230 of FIG. 2 is not limited to any specific order, if practicable.

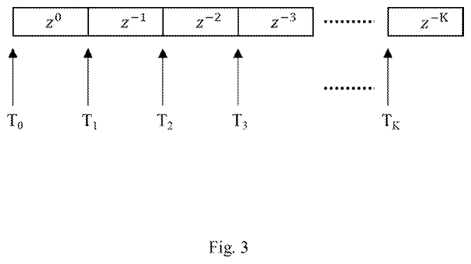

In regard to the embodiments of FIGS. 1-2, the PPG device 100 performs a Z-domain sampling operation through the time domain to obtain the M mixed-light digital value(s) and the N ambient-light digital value(s) as shown in FIG. 3, wherein the definitions of the time domain and the Z domain are well known in this technical field. Given (M+N−1)=K, the transformation of the K-order digital filter (e.g., the digital filter 130 in FIG. 1) can be expressed with the following equation: c₁z⁰+c₂z⁻¹+c₃z⁻²+. . . +c_{K+1}z^{-K}, wherein c₁˜c_{K+1} are coefficients of the K-order digital filter and can be determined/adjusted according to the demand for implementations, and z⁰˜z^{K} are (K+1) digital values (i.e., the aforementioned M mixed-light digital value(s) and N ambient-light digital value(s) which amount to (M+N) digital values) generated by the K-order digital filter in the sequence of sampling time points (i.e., T₀, T₁, T₂, T₃, . . . , and T_{K} in FIG. 3). In an exemplary implementation, provided that “M=1, N=2, and the (M+N) digital values are in the following sequence: a mixed-light digital value, an ambient-light digital value, and an ambient-light digital value”, the K-order (i.e., (M+N−1) order) digital filter is a 2-order digital filter, the transformation of the 2-order filter can be expressed with the equation “1z⁰−2z⁻²”, and the 2-order digital filter processes the three digital values z⁰, z⁻¹, and z⁻² to generate the output value. It is noted that the coefficients “+1, −2, +1” of the above-mentioned equation are predetermined and/or adjustable according to the demand for implementations. In an exemplary implementation, provided that “M=2, N=2, and the (M+N) digital values are in the following sequence: a mixed-light digital value, an ambient-light digital value, an ambient-light digital value, and a mixed-light digital value”, the K-order digital filter is a 3-order digital filter, the transformation of the 3-order filter can be expressed with the equation “1z⁰−3z⁻¹+3z⁻²−z⁻³”, and the 3-order digital filter processes the four digital values z⁰, z⁻¹, z⁻², and z⁻³ to generate the output value. It is noted that the coefficients “+1, −3, +3, −1” of the above-mentioned equation are predetermined and/or adjustable according to the demand for implementations.

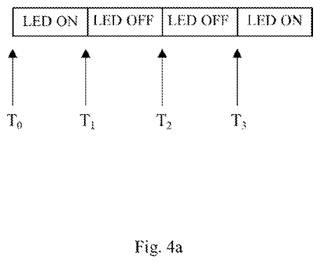

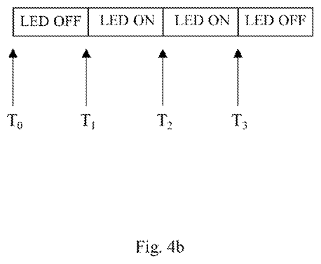

In an exemplary implementation of the embodiment of FIG. 2, the M is equal to the N, and each of the M and the N is greater than one. In this implementation, at least one ambient-light digital value of the N ambient-light digital values is between two closest mixed-light digital values of the M mixed-light digital values on the timeline due to the control of a controllable light source (e.g., at least one LED) as shown in FIG. 4a, or at least one mixed-light digital value of the M mixed-light digital values is between two closest ambient-light digital values of the N ambient-light digital values on the timeline due to the control of the controllable light source as shown in FIG. 4b. In FIGS. 4a-4b, “LED ON” denotes a mixed-light digital value, “LED OFF” denotes an ambient-light digital value, and M=N=2. It is noted that the term “two closest mixed-light digital values” implies that no other mixed-light digital value is between the two closest mixed-light digital values.

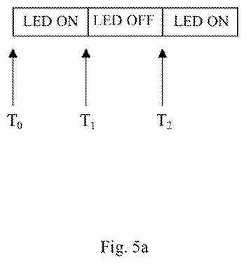

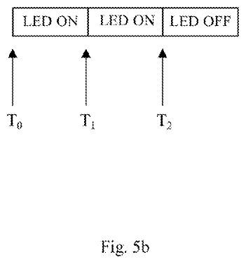

In an exemplary implementation of the embodiment of FIG. 2, the M is not equal to the N, and the M is greater than one. In this implementation, at least one ambient-light digital value of the N ambient-light digital value(s) is between two closest mixed-light digital values of the M mixed-light digital values on the timeline due to the control of a controllable light source as shown in FIG. 5a, or the M mixed-light digital values are consecutive on the timeline as shown in FIG. 5b. In FIGS. 5a-5b, “LED ON” denotes a mixed-light digital value, “LED OFF” denotes an ambient-light digital value, M=2, and N=1.

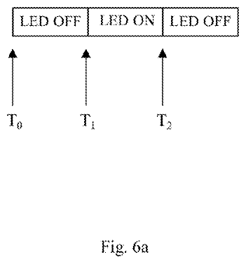

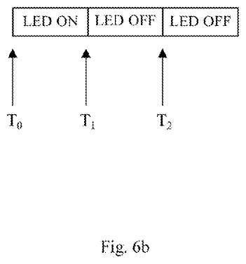

In an exemplary implementation of the embodiment of FIG. 2, the M is not equal to the N, and the N is greater than one. In this implementation, at least one mixed-light digital value of the M mixed-light digital value(s) is between two closest ambient-light digital values of the N ambient-light digital values on the timeline due to the control of a controllable light source as shown in FIG. 6a, or the N ambient-light digital values are consecutive on the timeline as shown in FIG. 6b. In FIGS. 6a-6b, “LED ON” denotes a mixed-light digital value, “LED OFF” denotes an ambient-light digital value, M=1, and N=2. It is noted that the term “two closest ambient-light digital values” implies that no other ambient-light digital value is between the two closest ambient-light digital values.

Since those having ordinary skill in the art can derive more examples from the aforementioned exemplary implementations, repeated and redundant description is omitted here.

It is noted that people having ordinary skill in the art can selectively use some or all of the features of any embodiment in this specification or selectively use some or all of the features of multiple embodiments in this specification to implement the present invention as long as such implementation is practicable; in other words, the way to implement the present invention can be flexible based on the present disclosure.

To sum up, the digital filtering method of the present disclosure is for a PPG device. The method can easily eliminate ambient light in a multi-order filtering manner, and is simple and cost-effective.

The aforementioned descriptions represent merely the preferred embodiments of the present invention, without any intention to limit the scope of the present invention thereto. Various equivalent changes, alterations, or modifications based on the claims of the present invention are all consequently viewed as being embraced by the scope of the present invention.

What is claimed is:

1. A digital filtering method for a photoplethysmography (PPG) device, wherein the PPG device is configured to sample a mixed-light signal M time(s) in turn to obtain M mixed-light digital value(s) and is configured to sample an ambient-light signal N time(s) in turn to obtain N ambient-light digital value(s), both the M and the N are positive integers, each of the M mixed-light digital value(s) includes a controllable-light component and an ambient-light component, and the digital filtering method comprises following steps:
preparing a digital filter, wherein a number of an order of the digital filter is (M+N−1);
using the digital filter to multiply the M mixed-light digital value(s) by M coefficient(s) respectively in accordance with a sequence of the M mixed-light digital value(s) being obtained, and thereby generating M value(s);
using the digital filter to multiply the N ambient-light digital value(s) by N coefficient(s) respectively in accordance with a sequence of the N ambient-light digital value(s) being obtained, and thereby generating N value(s); and
using the digital filter to add up the M value(s) and the N value(s), and thereby generating an output value.

2. The digital filtering method of claim 1, wherein the digital filter is a high pass filter.

3. The digital filtering method of claim 1, wherein the M coefficient(s) is/are predetermined and/or the N coefficient(s) is/are predetermined.

4. The digital filtering method of claim 1, wherein the M coefficient(s) is/are adjustable and/or the N coefficient(s) is/are adjustable.

5. The digital filtering method of claim 1, wherein the M coefficient(s) is/are of a first sign, the N coefficient(s) is/are of a second sign, the first sign is a positive sign or a negative sign, and the second sign is opposite to the first sign.

6. The digital filtering method of claim 1, wherein the M is equal to the N.

7. The digital filtering method of claim 6, wherein each of the M and the N is greater than one; on a timeline, at least one ambient-light digital value of the N ambient-light digital values is between two closest mixed-light digital values of the M mixed-light digital values, or at least one mixed-light digital value of the M mixed-light digital values is between two closest ambient-light digital values of the N ambient-light digital values.

8. The digital filtering method of claim 1, wherein the M is not equal to the N.

9. The digital filtering method of claim 8, wherein the M is greater than one; on a timeline, at least one ambient-light digital value of the N ambient-light digital value(s) is between two closest mixed-light digital values of the M mixed-light digital values, or the M mixed-light digital values are consecutive.

10. The digital filtering method of claim 8, wherein the N is greater than one; on a timeline, at least one mixed-light digital value of the M mixed-light digital value(s) is between two closest ambient-light digital values of the N ambient-light digital values, or the N ambient-light digital values are consecutive.
